(19) United States
(12) Patent Application Publication
(10) Pub. No.: US 2008/0082057 A1
(43) Pub. Date: Apr. 3, 2008

(54) Method and apparatus for diagnosing meibomian gland dysfunction

(76) Inventors: Donald R. Korb, Boston, MA (US); Benjamin T. Gravely, Raleigh, NC (US); John M. Jans, Hillsborough, NC (US); Keith H. Gausmann, Cary, NC (US)

Correspondence Address: Robert G. Rosenthal, Suite 200, 5856 Faringdon Place, Raleigh, NC, 27609, US

(21) Appl. No.: 11/541,240

(22) Filed: Sep. 29, 2006

(51) Int. Cl. A61M 35/00

(52) U.S. Cl. 604/289

(57) ABSTRACT

An apparatus for testing meibomian glands in the eyelids of humans for meibomian gland disease is provided. The apparatus comprises a handle, and a probe tip mounted for movement relative to said handle such that when the probe tip is placed against the eyelid and compressive force is applied, movement of the handle a preselected distance replicates the force required for natural expression of secretion from the meibomian gland. Movement of the handle relative to the probe tip the preselected distance exerts a force of between about 5 grams per 30 mm² and 40 grams per 30 mm² and preferably a force of approximately 15 grams per 30 mm². An indicator means selected from the group consisting of auditory, visual and tactile signals is provided and when activated alerts the clinician that the required force has been reached.

According to the method of the present invention a probe tip is located in contacting relation with the eyelid, the probe tip being connected to a handle. When pressure is exerted on the eyelid by pressing it against the eyelid, a preselected pressure is exerted thereon. In another embodiment of the invention, a handle having a probe tip is located on the eyelid, the probe tip is mounted for movement relative to said handle such that when the probe tip is placed against the eyelid and compressive force is applied, movement of the handle a preselected distance replicates the force required for natural expression of secretion from the meibomian gland

### FIELD OF THE INVENTION

This invention relates generally to the field of ophthalmology and more particularly diagnosing and treating dry eye disease as it relates to the health of the meibomian glands.

### BACKGROUND OF THE INVENTION

The human body contains a number of glands including the lacrimal and meibomian glands of the eye, the sebaceous or pilo-sebaceous hair glands on the face and underarms, and the mammary glands in the breasts. These glands may malfunction due to age, irritation, environmental conditions, cellular debris, inflammation, hormonal imbalance and other causes. One common disease state of the eyelid glands is the restriction or stoppage of the natural flow of fluid out of the gland caused by an obstruction.

In the human eye, the tear film covering the ocular surfaces is composed of three layers. The innermost layer in contact with the ocular surface is the mucus layer compromised of many mucins. The middle layer comprising the bulk of the tear film is the aqueous layer, and the outermost layer is a thin (less than 250 nm) layer comprised of many lipids known as “meibum” or “sebum”. The sebum is secreted ty the meibomian glands, enlarged specialized sebaceous-type glands (hence, the use of “sebum” to describe the secretion) located on both the upper and lower eye lids, with orifices designed to discharge the lipid secretions onto the lid margins, thus forming the lipid layer of the tear film. The typical upper eyelid has about 25 meibomian glands and the lower eyelid has about 20 meibomian glands, which are somewhat larger than those located in the upper lid and which therefore contribute a greater amount of these lipid secretions. The meibomian gland comprises various sac-like acini which discharge the secretion into the main central duct of the gland. The secretion then passes into the orifices which are surrounded by smooth muscle tissue and the muscle of Riolan which are presumed to aid in the expression of sebum. The meibomian gland orifices open onto the lid margin at and around the junction of the inner mucous membrane and the outer skin of the eyelids' mucocutaneous junction.

Specifically, each meibomian gland has a straight long central duct lined with four epithelial layers on the inner surface of the duct. Along the length of the central duct there are multiple lateral out-pouching structures, termed acini, where the secretion of the gland is manufactured. The inner lining of each acinus differs from the main central duct in that these specialized cells provide the secretions of the meibomian gland. The secretions flow from each acinus to the duct. While it has not been established with certainty, there appears to be a valve system between each acinus and the central duct to retain the secretion until it is required, at which time it is discharged in to the central duct. The meibomian secretion is then stored in the central duct and is released through the orifice of each gland onto the lid margin. Blinking and the squeezing action of the muscle of Riolan surrounding the meibomian glands are thought to be the primary mechanisms to open the orifice for the release of secretion from the meibomian gland.

Blinking causes the upper lid to pull a sheet of the lipids secreted by the meibomian glands upward over the other two layers of the tear film, thus forming a type of protective coating which limits the rate at which the underlying layers evaporate. Thus, it will be seen that a defective lipid layer or an incorrect quantity of such lipids can result in accelerated evaporation of the aqueous layer which, in turn, causes symptoms such as itchiness, burning, irritation, and dryness, which are collectively referred to as “dry eye”.

Dry eye states have many etiologies. A common cause of common dry eye states is the condition known as “meibomian gland dysfunction” (MGD), a disorder where the glands are obstructed or occluded. As employed herein the terms “occluded” and “obstruction” as they relate to meibomian gland dysfunction are defined as partially or completely blocked or plugged meibomian glands, or any component thereof, having a solid, semi-solid or thickened congealed secretion and/or plug, leading to a compromise, or more specifically, a decrease or cessation of secretion. Also with a reduced or limited secretion the meibomian gland may be compromised by the occluded or obstructive condition as may be evidenced by a yellowish color indicating a possible infection state, or may be otherwise compromised so that the resulting protective lipid protective film is not adequate.

Meibomitis, an inflammation of the meibomian glands leading to their dysfunction, is usually accompanied by blepharitis (inflammation of the lids). Meibomian gland dysfunction may accompany meibomitis, or meibomian gland dysfunction may be present without obvious lid inflammation. Meibomian gland dysfunction is frequently the result of keratotic obstructions which partially or completely block the meibomian gland orifices and/or the central duct (canal) of the gland, or possibly the acini or acini valves (assuming they do in fact exist) or the acini's junction with the central duct. Such obstructions compromise the secretory functions of the individual meibomian glands. More particularly, these keratotic obstructions are comprised of bacteria, sebaceous ground substance, dead, and/or desquamated epithelial cells, see, Korb et al., Meibomian Gland Dysfunction and Contact Lens Intolerance, Journal of the Optometric Association, Vol. 51, Number 3, (1980), pp. 243-251. While meibomitis is obvious by inspection of the external lids, meibomian gland dysfunction may not be obvious even when examined with the magnification of the slit-lamp biomicroscope, since there may not be external signs or the external signs may be so minimal that they are overlooked. The external signs of obstructive meibomian gland dysfunction may be limited to subtle alterations of the epithelium of the lid margins over the orifices, and pouting of the orifices of the glands with congealed material acting as obstructions. The external signs of obstructive meibomian gland dysfunction in severe to very sever instances may be obvious, and include serration and distortion of the shape of the lid margins. While inflammation is not usually present with mild to moderate obstructive meibomian gland dysfunction, with sever to very sever instances of obstructive meibomian gland dysfunction, inflammation may be present.

Hormonal changes, which occur during menopause and particularly changing estrogen levels, can result in thickening of the oils secreted by the meibomian glands which results in clogged gland orifices. Further, decreased estrogen levels may also enhance conditions under which staphylococcal bacteria can proliferate. This can cause migration of the bacteria into the glands, thus resulting in a decreased secretion rate.

When the flow of secretions from the meibomian gland is restricted due to the existence of an obstruction, cells on the eyelid margin have been observed to grow over the gland orifice thus further restricting sebum flow and exacerbating the dry eye condition. Additional factors which may cause or exacerbate meibomian gland dysfunction include, age, contact lens wear and hygiene, cosmetic use, or other illness, including diabetes.

The state of an individual meibomian gland can vary from optimal, where clear meibomian fluid is produced; to mild or moderate meibomian gland dysfunction where milky fluid or inspissated or creamy secretion is produced; to total blockage where no secretion of any sort can be obtained (see Korb, et al., “Increase in Tear Film Lipid Layer Thickness Following Treatment of Meibomian Gland Dysfunction”, Lacrimal Gland, tear Film, ad Dry Eye Syndromes, pp. 293-298, Edited by D. A. Sullivan, Plenum Press, New York (1994)). Significant chemical changes of the meibomian gland secretions occur with meibomian gland dysfunction and consequently, the composition of the naturally occurring tear film is altered, which in turn, contributes to ocular disease which is generally known as “dry eye”.

While the tear film operates as a singular entity and all of the layers thereof are important, the lipid layer, which is secreted from the meibomian glands, is of particular significance as it functions to slow the evaporation of the underlying layers and to lubricate the eyelid during blinking both of which prevent dry eye and epitheliopathies.

Thus, to summarize, the meibomian glands of mammalian (e.g., human) eyelids secrete oils that assist in preventing evaporation of the underlying tear film and provide lubrication to the eye. These glands can become blocked or plugged by various mechanisms leading to excess evaporation of the tear film, compromised lubrication and so-called “dry eye syndrome”. While not the only cause, meibomian gland dysfunction (MGD) is known to be a major cause of dry eye syndrome. The disorder is characterized by a blockage of some sort within the meibomian glands or at their surface preventing normal lipid secretions from flowing. Such secretions serve multiple functions including retarding evaporation and lubricating the eye, hence their absence can cause dry eye syndrome. Obstructions or occlusions of the meibomian glands may be present at the orifice of the gland, the main channel of the gland, or possibly in other locations including the main channel of the gland which may be harrowed or blocked. It has been theorized that the acini of the glands may have valves at their junction with the main channel of the gland, and that these valves may be obstructed in some instances leading to reduced or blocked flow from the acini. These obstructions or occlusions may have various compositions.

In response to the foregoing, various treatment modalities have been developed in order to treat the dry eye condition, including drops which are intended to replicate and replace the natural tear film, pharmaceuticals which are intended to stimulate the tear producing cells, and various heating devices which are designed to assist in unclogging the meibomian glands by manual expression.

However, prior to implementing an appropriate treatment plan, the clinician must first determine whether some or all of the meibomian glands are properly secreting or are obstructed. If it is determined that the glands are obstructed, determining the degree of such obstruction is helpful in developing a treatment plan. Current practice in diagnosing whether a meibomian gland is obstructed involves the application of force, usually with the thumb or finger to the external lid surfaces overlying the meibomian gland. The terms “gentle” and “forceful” expression have been used to describe the magnitude of the force used for meibomian gland evaluation. Further, as used herein, when referring to a “gland” the plural is intended to be included therein and when the term “glands” is used, the singular is intended to be included as well. Observations suggest that the diagnosis of whether an individual meibomian gland is obstructed is a function of the amount of force applied to the gland. While meibomian gland disease is often painful, clinicians often apply a force to the eyelid which is greater than the minimum force required to diagnose whether or not an obstruction exists and thus, inflict unnecessary pain on the patient.

Thus, it is critical for the clinician to determine whether the meibomian gland(s) are obstructed with normal and forced blinking since it is blinking that is the dynamic force that expresses the secretions of the meibomian gland(s) from the duct and through the orifice on to the lid margins and the tear film. The goal is to utilize a controlled method to simulate the forces of the lids in the act of blinking to mimic the action of the lids and the blink to express the secretions from the meibomian gland(s), thus allowing the diagnosis of obstruction of the meibomian gland(s).

In view of the foregoing, it is an object of the present invention to overcome the drawbacks and deficiencies of the prior art.

Another object of the present invention is to provide a method of diagnosing meibomian gland obstruction that is not painful.

A further object of the present invention is to provide a method of diagnosing meibomian gland obstruction that employs the lowest possible amount of force on the gland to minimize patient pain.

A still further object of the present invention is to provide an apparatus for the effective diagnosis of meibomian gland disease by evaluating the status of the meibomian gland secretion upon the application of a force simulating that of the blink actions and resultant forces.

A still further object of the present invention is to provide an apparatus that will provide a feedback means for optimum diagnosis of meibomian gland obstruction.

Yet another object of the present invention is to provide an apparatus which is inexpensive and easy to use.

Still another object of the present invention is to provide an apparatus for use in conjunction with a treatment to unclog blocked meibomian glands.

### SUMMARY OF THE INVENTION

To accomplish the objects described above, there is provided an apparatus for testing meibomian glands in the eyelids of humans for meibomian gland disease comprising a handle and a probe tip mounted for movement relative to said handle such that when the probe tip is placed against the eyelid and compressive force is applied, movement of the handle a preselected distance replicates the force required for natural expression of secretion from the meibomian gland. Movement of the handle relative to the probe tip the preselected distance exerts a force of between about 5 grams per 30 mm² and 40 grams per 30 mm² and preferably a force of approximately 15 grams per 30 mm². An indicator means selected from the group consisting of auditory, visual and tactile signals is provided and when activated alerts the clinician that the required force has been reached.

According to the method of the present invention a probe tip is located in contacting relation with the eyelid, the probe tip being connected to a handle. When pressure is exerted on the eyelid by pressing it against the eyelid, a preselected pressure is exerted thereon. In another embodiment of the invention, a handle having a probe tip is located on the eyelid, the probe tip is mounted for movement relative to said handle such that when the probe tip is placed against the eyelid and compressive force is applied, movement of the handle a preselected distance replicates the force required for natural expression of secretion from the meibomian gland

### BRIEF DESCRIPTION OF THE DRAWINGS

Some of the objects of the invention having been stated, other objects will appear as the description proceeds when taken in connection with the accompanying drawings in which:

FIG. 1a is a perspective view of a first embodiment of the meibomian gland evaluation tool according to the present invention showing a small tip for evaluation of one or just a few glands.

FIG. 1b is a perspective view of the first embodiment of the meibomian gland evaluation tool according to the present invention showing a larger tip for evaluation of several glands.

FIG. 2 is a broken away side view of the first embodiment of the meibomian gland evaluation tool according to the present invention.

FIG. 3 is s perspective view of a second embodiment of the meibomian gland evaluation tool according to the present invention.

FIG. 4 is a broken away side view of the second embodiment of the meibomian gland evaluation tool according to the present invention.

FIG. 5 is a broken away side view of a third embodiment of the meibomian gland evaluation tool according to the present invention.

FIG. 6 is an exploded side view of a forth embodiment of the meibomian gland evaluation tool according to the present invention.

FIG. 7 is a broken away side view of a fifth embodiment of the meibomian gland evaluation tool according to the present invention.

FIG. 8 is broken away side view of a sixth embodiment of the meibomian gland evaluation tool according to the present invention.

### DETAILED DESCRIPTION OF THE ILLUSTRATED EMBODIMENTS

While the present invention will be described more fully herein after with reference to the accompanying drawings, in which a particular embodiment is shown, it is to be understood at the outset that persons skilled in the art may modify the invention herein described while still achieving the favorable results of this invention. Accordingly, the description which follows is to be understood as a broad teaching disclosure directed to persons of skill in the appropriate arts and not as liming upon the present invention.

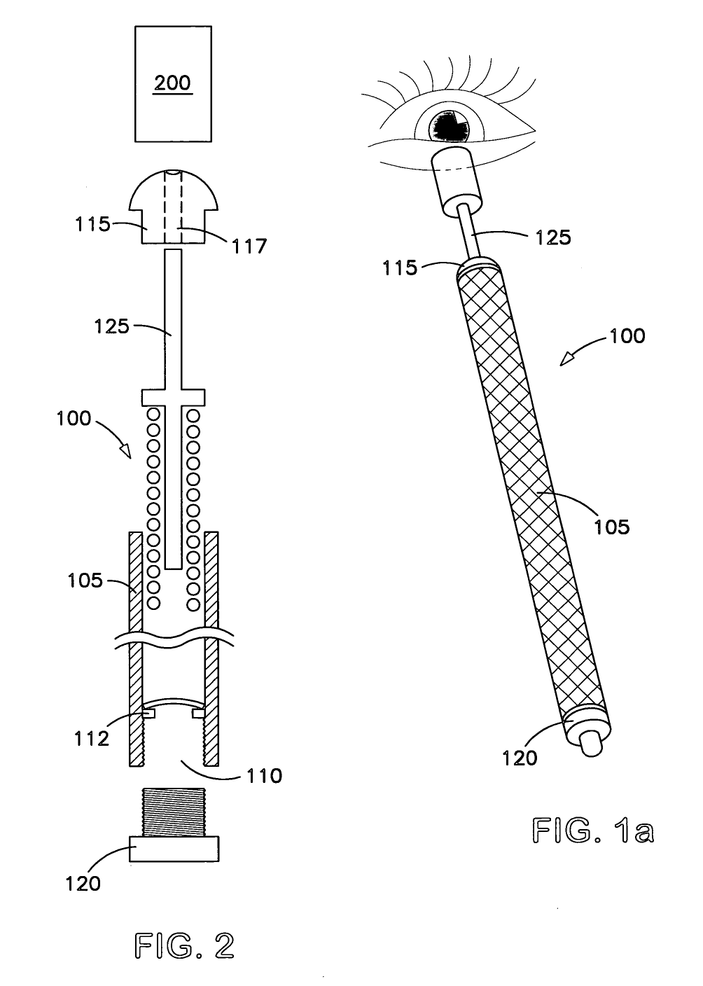

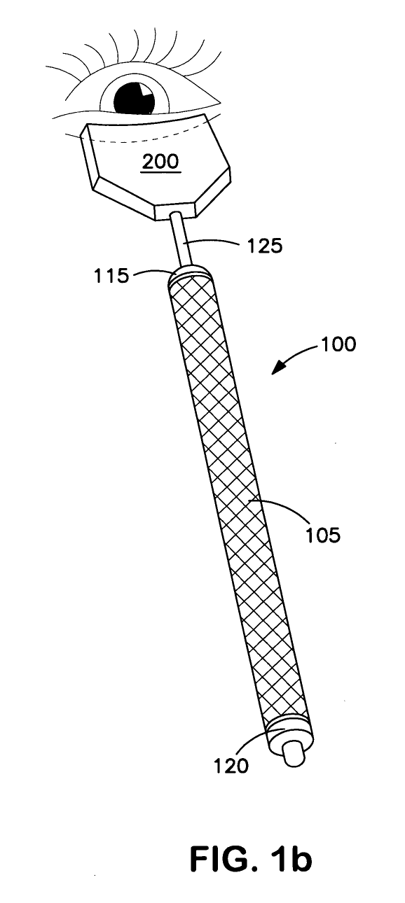

Referring to the figures and particularly FIGS. 1 and 2 in which a first embodiment of the of the invention is shown, the meibomian gland evaluation apparatus generally indicated at 100 comprises an elongate shaft or handle 105 having a bore 110 there through. Located at one end of handle 105 is an annulus 112 the purpose of which will become evident as the description proceeds. One end of handle 105 mounts a cap 115 having a bore there through 117 and the opposite end of handle 105 mounts a second end cap 120. The caps may be threaded, press fitted or otherwise connected, depending upon the particular fabrication technique and materials employed. For purposes of illustration only, cap 115 is press fitted and cap 120 is threaded. It will be noted that in most instances the present invention will be used in conjunction with a slit lamp and that the shaft should be of an appropriate dimension to be used in conjunction therewith.

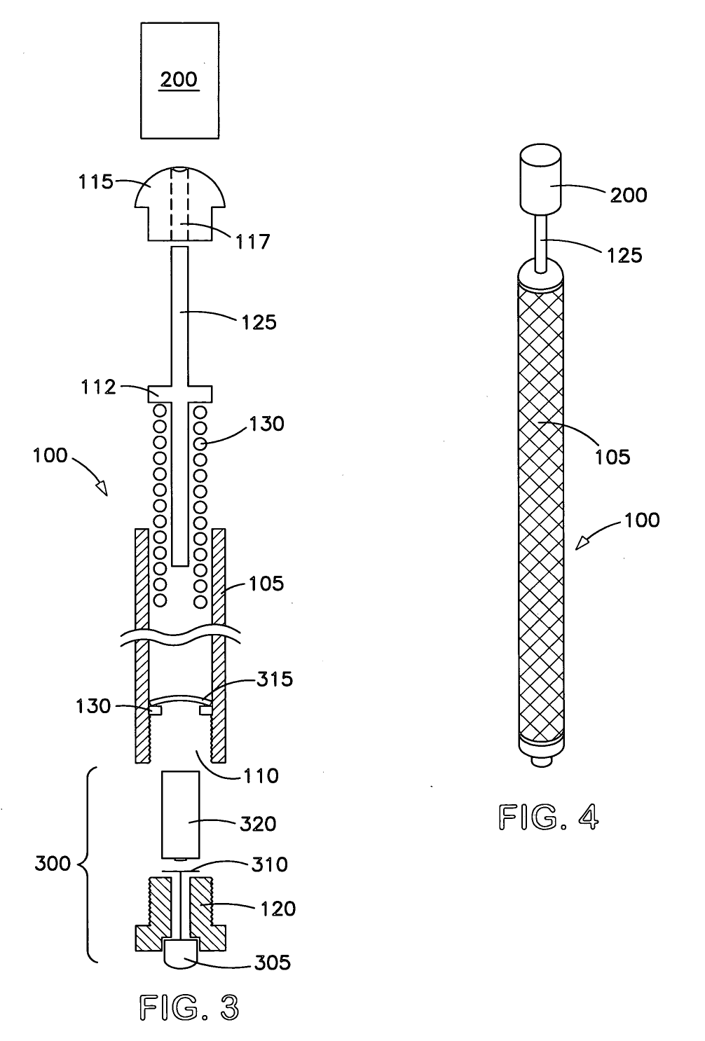

A probe tip 200 is mounted for longitudinal movement relative to the handle 105 such that when the probe tip 200 is placed against the eyelid and compressive force is applied, movement of the handle 105 a preselected distance replicates the approximate force required for natural expression of secretion from the meibomian gland. Testing has determined that this force is approximately 15 grams per 30 mm² however; depending upon the age, gender, race or other factors, this force may be between 10 grams per 30 mm² and 20 grams per 30 mm² or even up to 40 grams per mm² in some cases. Probe tip 200 is detachably connected to one end of a shaft 125 which is operatively associated with handle 105. Probe tip 200 is fabricated from a soft biocompatible material such as natural or synthetic rubber, Polyester® or other inert/non-allergenic or biocompatible materials, well known to those skilled in the art. As shown in FIGS. 2 and 4, probe tip 200 is cylindrical and may be dimensioned to as to overlie one or more meibomian glands. An alternate embodiment of probe tip 200 is shown connected to the handle as illustrated in FIG. 3 and in that embodiment is designed to test a larger section (approximately ¼) of the eyelid to simultaneously evaluate multiple meibomian glands for gland function. Probe tip 200 may be press fit, snapped or threaded on to the end of shaft 105. In addition, while the specification refers to a probe tip of 30 mm², it us to be understood that this size is exemplary of probe tips and that larger or smaller surface areas may be employed with equal efficacy, depending on the preference of the clinician, the equipment employed, the degree of obstruction present as well as other factors which may appear. Thus, the pressure per unit area to be tested will remain-substantially unchanged. For example, a probe tip of 15 mm² will have a pressure ranging between 5 grams per 15 mm² and 10 grams per 15 mm² according to the parameters set out above. Stated otherwise, under normal circumstances the probe tip will apply between ⅓ and ⅔ grams per mm².

Per FIG. 2, shaft 105 also includes an annulus 130 proximate the tip mounting end. As shown, shaft 125 is inserted within bore 110 for longitudinal movement. A helical spring 130 is operatively associated with shaft 105 and surrounds a section thereof, biasing the shaft out of the housing. Annulus 113 serves as a support or bearing surface for spring 130.

As illustrated in the second embodiment of the invention, shown in FIG. 3, the apparatus may also include an indicator means or indicator generally indicated at 300 for indicating when handle 105 has moved the preselected distance. The indicator means 300 may be selected from the group consisting of auditory, visual and tactile signals. Any of the just mentioned signal means may be employed so long as activation thereof does not significantly impact the force required to move the handle to ensure that the pressure delivered to the eyelid remains in the required range. The indicator means 300 comprises a visual indicator means or light emitting diode (LED) 305 mounted in end cap 120 such that the light emitting portion is at least partially external of the cap and the electrical leads 310 (schematically shown) extend down into the bore 110 and are connected to a battery contact plate 315 also within handle 105. A battery 320 is provided proximate the battery contact plate 315. The LED is activated by movement of the handle 105 which causes the end of shaft 125 to complete the electrical circuit and illuminate the LED. Movement of the handle 105 away from the eyelid opens the electrical circuit and turns off the LED. Circuits of this nature are well known the art and a detailed discussion thereof is not deemed necessary. Buzzers, vibrators or other indicator means may also be employed as visual indicator means.

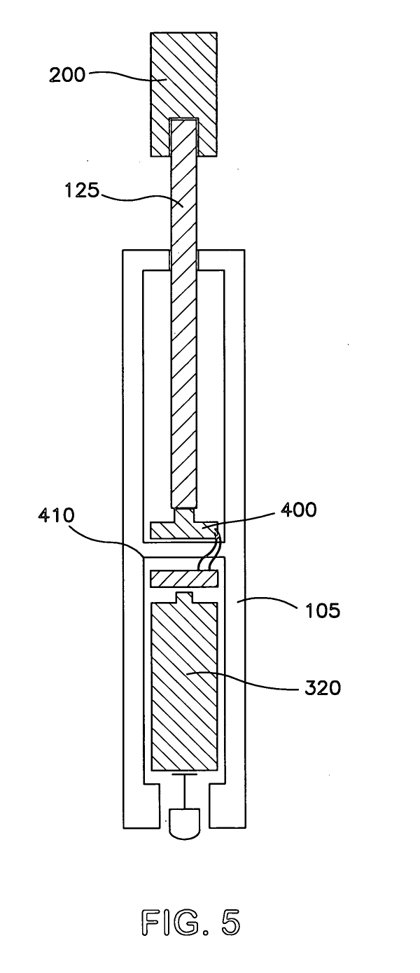

In the embodiment of FIG. 5, the meibomian gland evaluation tool 100 is generally similar to the previously described embodiment except that the shaft 125 is substantially stationary and the means for sensing when the preselected pressure has been reached comprises a piezo-electric or other similar strain gauge device 400 in combination with an amplification circuit 410 (shown schematically) and which is well known to those skilled in the art. When the preselected pressure has been exerted on the eyelid, the amplifier is activated and the indicator means 300 is triggered. It is believed that this embodiment will be produced using molding techniques wherein the cylindrical handle 105 will be produced in two longitudinal halves and will be press fit together.

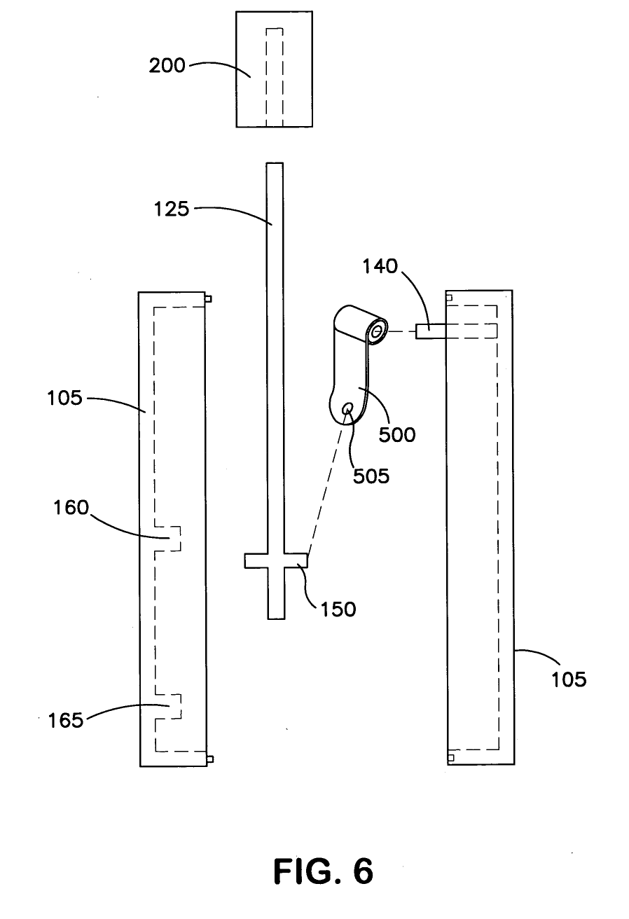

In the embodiment of FIG. 6, the preselected pressure is supplied by a spring means or constant force spring 500 which has a spring constant selected to deliver the preselected pressure to the eyelid. The constant force spring is coiled and has a connection opening 505 at the outer end. In this embodiment, it is again believed that the handle 105 will be molded in two opposing longitudinal sections that will be press fit together. One half of handle 105 is provided with upper stop 160 and lower stop 165 in the form of protuberances extending into the bore 110 which operate to limit the travel of probe shaft 125, as will be described more fully herein below. In addition, one side of the handle includes a tang 140 extending from the inner handle wall towards the center of the bore 110. The tang 140 should be of a diameter to receive the opening in the center of constant force spring 500 and should be of a length sufficient to maintain the spring in place when the two halves of the handle are connected together. The other end of spring 500 is connected to a tang 150 located on shaft 125. In the “at rest” state of this embodiment, the spring 500 is in the coiled position and tang 150 is in contact with upper stop 160. Pressure exerted on the probe tip by movement of the handle 105 causes the spring to uncoil until tang 150 contacts lower stop 165. An indicator means is not provided as the constant force is delivered merely by unwinding spring 500. Further, it is believed that the clinician will sense when the shaft has reached its maximum path of travel when tang 150 contacts lower stop 165, but the indicator means which could buzz, flash, vibrate, or illuminate when shaft 125 is in the operating range between stops 160 and 165 previously described, could also be included with this embodiment of the invention.

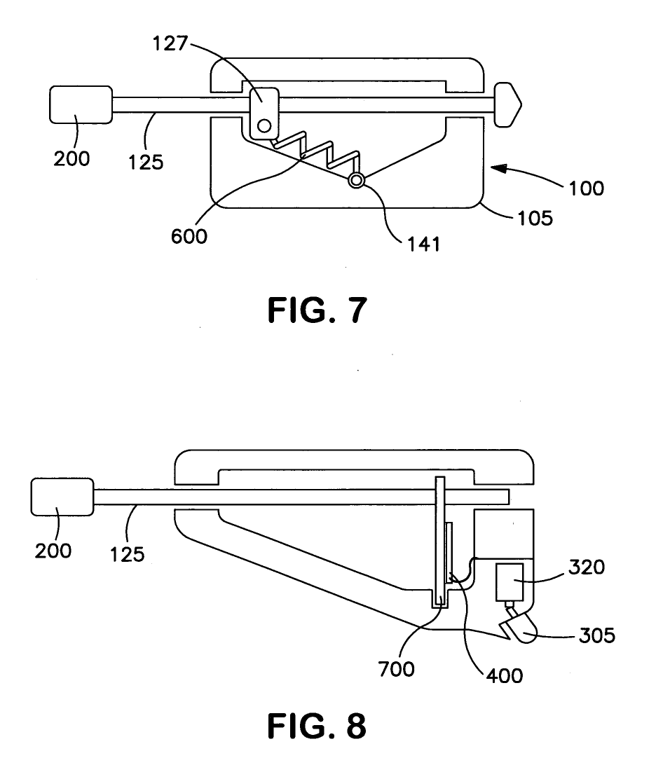

FIG. 7 illustrates an alternate embodiment of the invention wherein the handle shape is rectangular and box-like. Shaft 125 includes a mounting bracket 127 to which one end of an over center spring or compression spring 600 is connected. As a compression spring is normally expanded, the first end rests in a cavity or pocket 141 in handle 105. The opposite end of spring 600 is connected to the handle by means of a pin or tang formed in the handle housing and the shaft is biased in the extended or outward position. Pressure on the eyelid acts to compress the spring 600. When shaft 105 is pushed past the center position 127 or 141, shaft 105 will retract away from the eyelid. The device may be reset by pressing a reset button (not shown), but well known to those skilled in the art.

FIG. 8 illustrates another embodiment of the meibomian gland evaluation tool 100 wherein the shaft 125 is connected to handle 105 with a cantilever beam 700. A piezo-electric transducer or strain gauge 400 together with actuator circuitry identical to that discussed in connection with the embodiment of FIG. 5. Pressure on probe tip 200 causes strain gauge to output a signal proportional to the applied pressure. When the preselected pressure has been reached, the actuator circuit activates LED 305.

In operation the clinician selects the handle 100 having the desired probe tip 200 or mounts the desired probe tip 200 at the end of shaft 125. The probe tip 200 is then placed on the external surface of that section of the eyelid to be tested for meibomian gland function. The clinician also equips themselves with the proper equipment (appropriate magnification from a hand held lens, head magnifier, slit lamp, microscope etc.) to be able to observe the meibomian gland orifice(s) in order to monitor whether secretion is expressed from the orifices of the meibomian gland with the application and the continuation of the compressive force. A compressive force in the form of gentle pressure is exerted upon the eyelid by pressing the handle 105 towards the eyelid which compresses spring 130. Just prior to the end of shaft 125 makes contact with the battery contact plate 315, the force of 15 grams per 30 mm² is reached in a user independent manner and the clinician observes whether the meibomian gland is properly secreting or not, and the type of abnormality of secretion varying from total obstruction to compromised secretion. The apparatus is designed so that just prior to activation of the indicator means, the cumulative force or energy stored in the spring is substantially equivalent to the force required for natural meibomian gland secretion. Of course, the other indicator means are actuated in the aforementioned manner as well.

In another embodiment (not shown) of the meibomian gland evaluation tool 100, the shaft 125 may be connected to handle 105 which is attached to a coil spring of constant force which rotates and provides force either directly on the eyelid or by pushing a linear rod attached to the handle 10S which applies force on the eyelid.

It will be noted that this apparatus of the present invention may be fabricated as a disposable, single use item primarily from plastic materials, or alternatively, may be fabricated as a multiple use probe with disposable tips, in which case that portion of the device that is re-used will be fabricated from materials of sufficient durability to withstand repeated autoclaving and/or alcohol sterilization, as appropriate for the materials chosen.

The foregoing embodiments and examples are to be considered illustrative rather than restrictive of the invention, and those modifications which come within the meaning and range of equivalence of the claims are to include therein.

That which is claimed is:

1. An apparatus for testing meibomian glands in the eyelids of humans for meibomian gland obstruction comprising:
a handle;
a probe tip operatively associated with said handle;
means for exerting a preselected pressure on the eyelid when the probe tip is pressed against the eyelid.

2. The apparatus according to claim 1 further including a shaft operatively associated with said handle and being connected for movement with respect to said handle and further, wherein one end of said shaft mounts said probe tip.

3. The apparatus according to claim 2 wherein said shaft is mounted for longitudinal movement within said handle.

4. The apparatus according to claim 3 wherein said means for exerting a preselected pressure comprises a constant force spring operatively associated with said shaft such that movement of the shaft exerts the constant preselected pressure upon the eyelid.

5. The apparatus according to claim 4 wherein the preselected pressure is between about 10 grams per 30 mm² and 20 grams per 30 mm².

6. The apparatus according to claim 1 wherein the preselected pressure is approximately about 15 grams per 30 mm².

7. The apparatus according to claim 5 wherein the preselected pressure is between about 5 grams per 30 mm² and 40 grams per 30 mm².

8. The apparatus according to claim 5 wherein the preselected pressure is approximately about 15 grams per 30 mm².

9. An apparatus for testing meibomian glands in the eyelids of humans for meibomian gland obstruction comprising:
a handle;
a probe tip operatively associated with said handle and adapted to be placed in contact with the eyelid;
a sensor means operatively associated with said probe tip for sensing when a preselected pressure has been exerted upon the eyelid by the probe tip when the probe tip is placed in contact with the eyelid and pressure is exerted thereon and being adapted to produce an output signal; and
indicator means for indicating when the preselected pressure has been exerted on the eyelid said indicator means being activated by said sensor means output signal.

10. The apparatus according to claim 9 wherein the preselected pressure is between about 5 grams per 40 mm² and 20 grams per 30 mm².

11. The apparatus according to claim 9 wherein the preselected pressure is approximately about 15 grams per 30 mm².

12. The apparatus according to claim 9 further including a shaft operatively associated with said handle and being connected for movement relative to said handle and further, wherein one end of said shaft mounts said probe tip.

13. The apparatus according to claim 12 wherein said shaft is mounted for longitudinal movement within said handle.

14. The apparatus according to claim 9 wherein said sensor means is selected from the group consisting of electrical contact, piezo-electric and strain gauge.

15. The apparatus according to claim 9 wherein said indicator means is selected from the group consisting of auditory, visual and tactile signals.

16. The apparatus according to claim 13 wherein the preselected pressure is between about 5 grams per 30 mm² and 40 grams per 30 mm².

17. The apparatus according to claim 13 wherein the preselected pressure is approximately about 15 grams per 30 mm².

18. The apparatus according to claim 13 further including a spring means operatively associated with said shaft and biasing said shaft out of said handle.

19. The apparatus according to claim 13 wherein said spring means is a constant force spring.

20. The apparatus according to claim 18 wherein movement of said handle a preselected distance causes compression of said spring means and in the tip exerting the preselected pressure on the meibomian gland and the sensor means activating the indicator means.

21. An apparatus for testing meibomian glands in the eyelids of humans for meibomian gland obstruction comprising:
a handle;
a probe tip operatively associated with said handle such that when the probe tip is placed against the eyelid and a preselected compressive pressure is applied, the pressure required for natural expression of secretion from the meibomian gland is replicated and a diagnosis concerning meibomian gland obstruction may be made.

22. The apparatus according to claim 21 wherein the preselected pressure applied to the eyelid is between about 5 grams per 30 mm² and 40 grams per 30 mm².

23. The apparatus according to claim 21 wherein the preselected pressure applied to the eyelid approximately 15 grams per 30 mm².

24. The apparatus according to claim 21 wherein the probe tip is connected to a shaft which is operatively associated with said handle and further, wherein said shaft is biased outward such that movement of said handle towards the meibomian gland exerts pressure upon the meibomian gland.

25. The apparatus according to claim 24 further including a spring operatively associated with said shaft and biasing said shaft outward.

26. The apparatus according to claim 23 further including indicator means for indicating when the preselected pressure is exerted on the eyelid.

27. The apparatus according to claim 26 wherein the indicator means is selected from the group consisting of auditory, visual and tactile signals.

28. A method for testing meibomian glands in the eyelids of humans for meibomian gland obstruction comprising:
locating a probe tip operatively associated with a handle in contacting relation with the eyelid; and
exerting a preselected pressure on the eyelid when the probe tip is pressed against the eyelid.

29. The method according to claim 28 further including a shaft operatively associated with the handle and being connected for movement with respect to the handle and further, wherein one end of the shaft mounts the probe tip.

30. The method according to claim 29 wherein the shaft is mounted for longitudinal movement within the handle.

31. The method according to claim 30 wherein the means for exerting a preselected pressure comprises a constant force spring operatively associates with the shaft such that movement of the shaft exerts the constant preselected pressure upon the eyelid.

32. The method according to claim 31 wherein the preselected pressure is between about 5 grams per 30 mm² and 40 grams per 30 mm².

33. The method according to claim 28 wherein the preselected pressure is approximately about 15 grams per 30 mm².

34. The method according to claim 32 wherein the preselected pressure is between about 10 grams per 30 mm² and 20 grams per 30 mm².

35. The method according to claim 32 wherein the preselected pressure is approximately about 15 grams per 30 mm².

36. A method for testing meibomian glands in the eyelids of humans for meibomian gland obstruction comprising:
locating a probe tip operatively associated with a handle and in contact with the eyelid;
sensing when a preselected pressure has been exerted upon the eyelid by the probe tip when the probe tip is placed in contact with the eyelid and pressure is exerted thereon; and
activating a user sensible signal indicating that the preselected pressure has been exerted on the eyelid.

37. The method according to claim 36 wherein the preselected pressure is between about 5 grams per 30 mm² and 40 grams per 30 mm².

38. The method according to claim 36 wherein the preselected pressure is approximately about 15 grams per 30 mm².

39. The method according to claim 36 further including a shaft operatively associated with the handle and being connected for movement relative to the handle and further, wherein one end of the shaft mounts said probe tip.

40. The method according to claim 39 wherein the shaft is mounted for longitudinal movement within the handle.

41. The method according to claim 36 wherein the sensor is selected from the group consisting of electrical contact, piezo-electric and strain gauge.

42. The method according to claim 36 wherein the user sensible signal is selected from the group consisting of auditory, visual and tactile signals.

43. The method according to claim 40 wherein the preselected pressure is between about 5 grams per 30 mm² and 40 grams per 30 mm².

44. The method according to claim 40 wherein the preselected pressure is approximately about 15 grams per 30 mm².

45. The method according to claim 40 further including a spring operatively associated with the shaft and biasing said shaft out of the handle.

46. The method according to claim 40 wherein the spring is a constant force spring.

47. The method according to claim 45 wherein movement of the handle a preselected distance causes compression of said spring means and in the tip exerting the preselected pressure on the meibomian gland and the sensor means activating the indicator means.

48. A method for testing meibomian glands in the eyelids of humans for meibomian gland obstruction comprising:
positioning a probe tip operatively associated with a handle such that when the probe tip is placed against the eyelid and a preselected compressive pressure is applied, the pressure required for natural expression of secretion from the meibomian gland is replicated and a diagnosis concerning meibomian gland obstruction may be made.

49. The method according to claim 48 wherein the preselected pressure applied to the eyelid is between about 5 grams per 30 mm² and 40 grams per 30 mm².

50. The method according to claim 48 wherein the preselected pressure applied to the eyelid approximately 15 grams per 30 mm².

51. The method according to claim 48 wherein the probe tip is connected to a shaft which is operatively associated with the handle and further, wherein the shaft is biased outward such that movement of the handle towards the meibomian gland exerts pressure upon the meibomian gland.

52. The method according to claim 51 further including a spring operatively associated with the shaft and biasing the shaft outward.

53. The method according to claim 50 further including an indicator for indicating when the preselected pressure is exerted on the eyelid.

54. The method according to claim 53 wherein the indicator is selected from the group consisting of auditory, visual and tactile signals.
